(19) United States
(12) Patent Application Publication
(10) Pub. No.: US 2024/0324995 A1
(43) Pub. Date: Oct. 3, 2024

(54) ULTRASOUND DEVICE COMPRISING POROUS GRAPHENE PREPARED FROM POLYIMIDE

(71) Applicant: GEORGE MASON UNIVERSITY, Fairfax, VA (US)

(72) Inventors: Parag Vijay Chitnis, Fairfax, VA (US); Pilgyu Kang, Fairfax, VA (US); Shirin Movaghgharnezhad, Fairfax, VA (US); Siddhartha Sikdar, Fairfax, VA (US)

(21) Appl. No.: 18/427,584

(22) Filed: Jan. 30, 2024

Related U.S. Application Data
(60) Provisional application No. 63/455,665, filed on Mar. 30, 2023.

(51) Int. Cl. A61B 8/00; A61L 31/06; A61L 31/08; A61L 31/14

(52) U.S. Cl. CPC A61B 8/4494; A61B 8/4444; A61L 31/06; A61L 31/084; A61L 31/146

(57) ABSTRACT

Ultrasound devices including wearable, patch-type ultrasound devices, which feature a porous graphene electrode prepared by graphitization of a polyimide precursor.

### CROSS-REFERENCE TO RELATED APPLICATIONS

This application claims priority to U.S. Provisional Application No. 63/455,665, filed Mar. 30, 2023, which is incorporated into this application by reference.

### STATEMENT OF GOVERNMENT SUPPORT

This invention was made with government support under W81XWH-21-1-0190 and W81XWH-22-9-0016 awarded by the United States Army Medical Research and Development Command. The government has certain rights in the invention.

### BACKGROUND

Ultrasonic images, known as sonograms, result from pulses of ultrasound sent into tissue using a probe. The pulses echo off tissues with different reflections and are recorded and displayed as an image. Ultrasound can create images of internal anatomical structures such as tendons, muscles, joints, blood vessels, internal organs, and the like. Ultrasound has several advantages over other diagnostic methods. It is non-invasive and provides images in real time. Ultrasound is also cost effective relative to other imaging techniques and does not use harmful ionizing radiation.

Currently, there are no suitable products that enable robust and objective muscle-level structural and functional assessments in ambulatory settings. An important aspect of rehabilitation following injury is the ability to perform dynamic assessment of movement through the synergistic assessment of functional and mechanical properties of the affected muscles, tendons, and associated soft tissue. In a clinical setting, such assessments are typically performed subjectively during physical examination of joint range of motion, strength, and functional task performance. Kinematic and kinetic evaluations using 3D motion capture and isokinetic dynamometry are quantitative extensions of routine clinical assessment, but these measures are limited to biomechanical laboratories. Muscle-level measurements can be made using surface electromyography (sEMG), but this is limited by poor signal to noise, lack of specificity in isolating deep-seated and overlying muscles, and difficulties in characterizing the resulting data.

### SUMMARY

The disclosed ultrasound device comprises a first electrode comprising porous graphene. The first electrode can be disposed within or on at least a portion of a substrate comprising a polyimide which has at least one aromatic ring. Within and in some aspects on the surface of the graphene electrode is a piezoelectric material. In some aspects, the device can further comprise a second electrode on top of at least a portion of the piezoelectric material.

The method of making the ultrasound device comprises graphitizing at least a portion of a substrate comprising a polyimide having at least one aromatic ring to form a first electrode comprising porous graphene disposed within or on the substrate; and applying a piezoelectric material to at least a portion of the first electrode. In some aspects, an additional second electrode can be applied to the piezoelectric material.

The ultrasound device is useful for a variety of applications. In one aspects, disclosed is a method of assessing an anatomical structure in a subject, comprising obtaining at least one ultrasound image from a disclosed ultrasound device applied to a target area of the subject.

### BRIEF DESCRIPTION OF THE DRAWINGS

The foregoing summary, as well as the following description of the disclosure, is better understood when read in conjunction with the appended drawings. For the purpose of illustrating the disclosure, the drawings illustrate some, but not all, alternative embodiments. This disclosure is not limited to the precise arrangements and instrumentalities shown. The following figures, which are incorporated into and constitute part of the specification, assist in explaining the principles of the disclosure.

FIG. 1 shows an example ultrasound device and its application as a wearable patch type device for assessing musculoskeletal activity and function of a subject during movement.

FIG. 2A depicts an example fabrication process for preparing a composite laser-induced graphene (LIG)/polyvinylidene fluoride-trifluoroethylene (PVDF-TrFE) (LIG/PVDF-TrFE composite) transducer.

FIG. 2B shows a photograph of a single element LIG/PVDF-TrFE transducer and a microscope image of a LIG/PVDF-TrFE 32-array transducer.

FIG. 2C shows photographs of LIG/PVDF-TrFE-based transducers having different patterns.

FIG. 3A shows a scanning electron microscopy (SEM) image of bare LIG.

FIG. 3B shows an SEM image of LIG having integrated PVDF-TrFE.

FIG. 3C shows a cross-sectional SEM image of the LIG/PVDF-TrFE.

FIG. 3D shows a Raman spectra of LIG.

FIG. 3E shows x-ray diffraction plots of PVDF-TrFE after annealing and poling, indicating the formation of a β phase.

FIG. 4A shows plots of capacitance of un-poled and poled LIG/PVDF-TrFE.

FIG. 4B shows plots of electrical impedance of LIG/PVDF-TrFE.

FIG. 4C shows a plot of output voltage of the LIG/PVDF-TrFE under a compression test over time.

FIG. 5A is a plot of pulse-echo signal from a 3.5-mm LIG/PVDF-TrFE-based ultrasound transducer.

FIG. 5B is a plot of the calculated frequency response of the 3.5-mm LIG/PVDF-TrFE-based ultrasound transducer.

FIG. 5C is a plot of pulse-echo signal from a comparative, 3.5-mm Ag-based ultrasound transducer.

### DETAILED DESCRIPTION

Musculoskeletal ultrasound has emerged as a promising new assessment technique for rehabilitation and biomechanics. Musculoskeletal ultrasound can provide spatially resolved quantitative and qualitative information about structure, composition, and dynamic function or dysfunction of multiple muscles. Musculoskeletal ultrasound applications have grown in recent decades with advances in image quality, resolution, and device portability. Musculoskeletal ultrasound is now the diagnostic standard for a number of conditions such as tendinopathy, ligament injuries, muscle tear, hematoma, rupture, and image-guided injections. However, the diagnostic criteria are currently based on subjective assessments of image features and are highly dependent on operator training and expertise.

Despite the promise of musculoskeletal ultrasound, the development of quantitative outcome measures is hampered by several practical challenges. Although ultrasound technology has advanced rapidly in recent years with the introduction of point of care ultrasound (POCUS) and hand-held ultrasound (HHUS), transducers continue to be designed for handheld operation, with significant operator dependence in acquiring and interpreting ultrasound images, limiting deployment in settings where trained operators are not available. Furthermore, the bulky form factor of handheld transducers prevents them from being readily used for continuous monitoring of muscle during gait or other functional movement, or as a biofeedback system for rehabilitation. Another challenge relates to sustaining adequate probe to skin contact and maintaining orientation of the probe relative to anatomical landmarks during dynamic examinations. A wearable hands-free ultrasound system for quantitative assessment of rehabilitation and recovery from injury in a clinical setting would address these limitations.

### I. Ultrasound Device

The disclosed ultrasound device in general comprises: (a) a porous graphene electrode (first electrode) disposed within or on at least a portion of a substrate comprising a polyimide which has at least one aromatic ring; and (b) a piezoelectric material disposed on the first electrode and within at least a portion of the porous graphene. In some aspects, the ultrasound device further comprises a top electrode or second electrode disposed on the piezoelectric material.

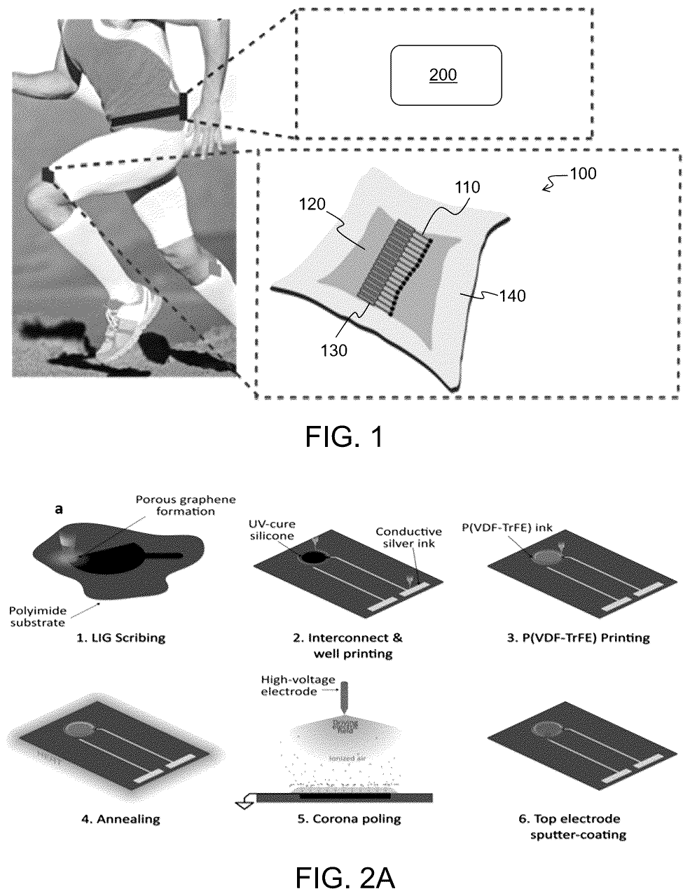

With reference to FIG. 1, in one example and non-limiting application of the device, the device can be configured as a wearable patch 100. The patch in some aspects can include an array of porous graphene based electrodes 110 formed by graphitization of an underlying polyimide substrate or layer 120. The piezoelectric material 130 can be applied to at least a portion of the graphene electrodes. Optionally, in some aspects, the device can include an underlying substrate 140, which can be adhesive, flexible, or elastic, and can be applied to a target area of a user's skin. A system comprising the device can further comprise an electronics component 200, in addition to other components, which enable collection and processing of one or more ultrasound images from the device.

### A. Substrate

The substrate of the ultrasound device may vary in composition depending on the desired application. In some aspects, the substrate can be flexible, elastic, or both. Flexible and elastic substrates are useful for example in wearable applications of the ultrasound device. For instance, the substrate may be placed against a user's skin, held close to the skin, or be incorporated in a piece of clothing, headwear, footwear, eyewear, accessory, blanket, band aid, bandage, or the like. Thus, in some aspects, the substrate can be made of a biocompatible material that will not irritate or otherwise damage the user's skin.

A variety of materials are suitable for the substrate. In one aspect, the substrate comprises a polymer, for example a flexible or elastic polymer. Non-limiting examples include the polyimide described below (which when graphitized yields the porous graphene electrode(s)), polyethylene terephthalate (PET), polyester, mylar, kapton, polytetrafluorethylene, surface modified polytetrafluoroethylene, expanded polytetrafluoroethylene, surface modified expanded polytetrafluoroethylene, low density polyethylene, medium density polyethylene, polyvinylidene, cellulose acetate, cellulose nitrate, polyimide, polydimethylsiloxane, SiO₂-PET, or SiNx-kapton. A polytetrafluroethylene type substrate can be surface modified to enable adhesion of deposited films by plasma treatment, sodium etching, or any other suitable method for improving adhesion. In some aspects, the substrate can include an adhesive on a skin-facing side of the substrate, to enhance adherence of the device to a user's body. Prior to use, the adhesive can be covered with a removable film layer according to methods known in the art.

The porous graphene electrode can be prepared by graphitizing the polyimide having at least one aromatic ring. Thus, in some aspects, the substrate may be the polyimide itself, which after graphitization comprises the porous graphene electrode layer disposed and in some aspects patterned within or on at least a portion of a surface thereof. In a further aspect, an underlying substrate layer can be used (i.e., below the graphene precursor polyimide layer), which can include for instance any of the polymeric materials described above. According to this aspect, the polyimide can be coated, deposited, or otherwise applied to an underlying substrate, followed by graphitization to form the porous graphene electrode(s). For example, the polyimide can be dissolved in a solvent such as a polar aprotic solvent (e.g., diamine, N,N-dimethylformamide, N,N-dimethylacetamide, N-methylpyrrolidone) and applied to a substrate layer at a temperature of 15-75° C.

In a further aspect, as discussed more below, a precursor to the polyimide, a corresponding polyamic acid, can initially be the substrate itself or be applied to an underlying substrate. The precursor can first be thermally imidized to the polyimide, followed by graphitization to form the porous graphene electrode(s) on or within at least a portion of a surface of the polyimide.

In one aspect, the substrate comprises a polyimide suitable for graphitization to form the porous graphene electrode layer, which has the structure of formula I or II:

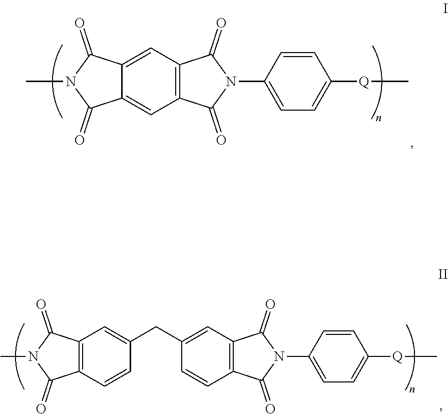

where n is an integer of at least two, and Q has the structure A or B:

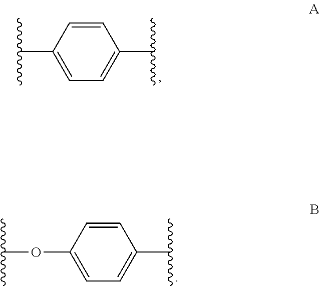 [SMILES: *c1ccc(*)cc1 ; *Oc1ccc(*)cc1]

Suitable non-limiting examples of polymers of formula I or II include:

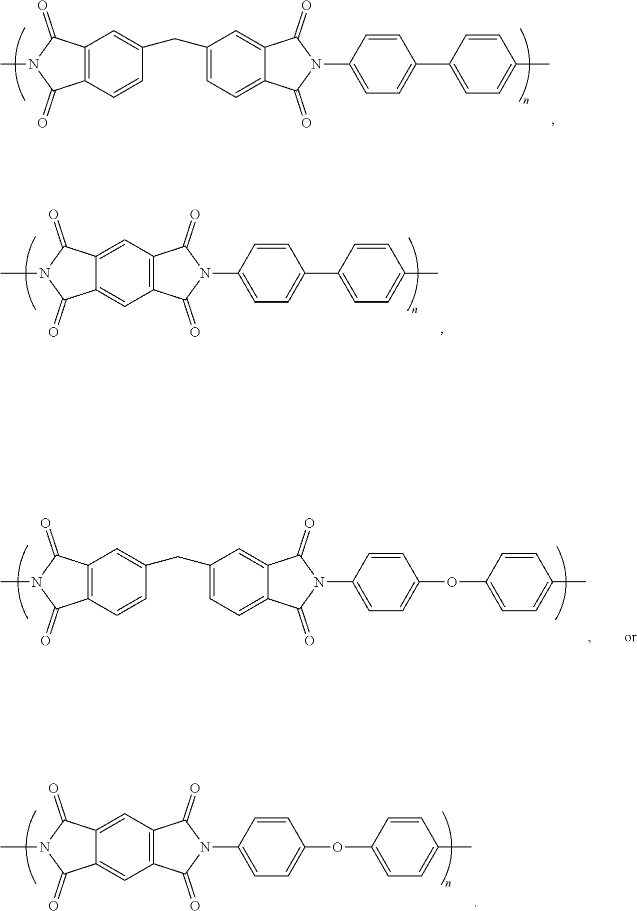

In one aspect, the polyimide has the formula:

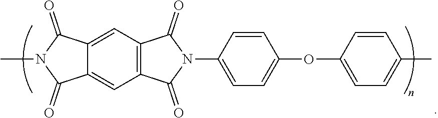

A variety of molecular weights beyond dimers are contemplated, typically only limited by the ability in some aspects to spin coat or otherwise solution coat or deposit the polyimide or a precursor thereof onto a substrate for imidization or graphitization. Thus, for instance, n values corresponding to molecular weights of up to 100,000 g/mol are contemplated, e.g., n values corresponding to a molecular weight of 5,000-100,000 g/mol, 5,000-80,000 g/mol, 5,000-60,000 g/mol, 5,000-50,000 g/mol, or 5,000-40,000 g/mol.

In one aspect, the polyimide can be prepared by thermal imidization of a precursor polyamic acid, including for example a precursor polyamic acid substrate (i.e., in situ thermal imidization), or a precursor polyamic acid layer that is deposited on an underlying substrate. In a further aspect, the polyimide can first be thermally imidized from the corresponding polyamic acid, and once formed, the polyimide can be formed into the substrate itself or applied to an underlying substrate layer.

The precursor polyamic acid can correspond to formula I or II in precursor form, with Q groups being the same as those described above:

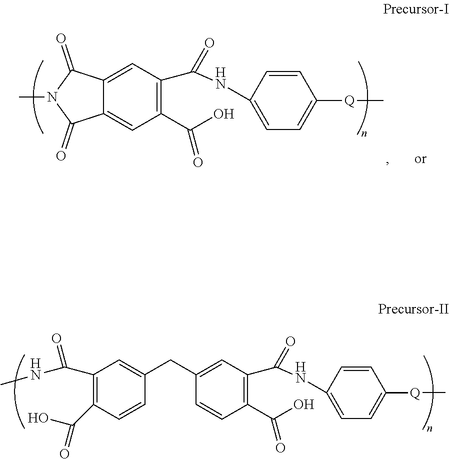

Accordingly, non-limiting examples of polyamic acids that are precursors to polyimides of formula I or II include:

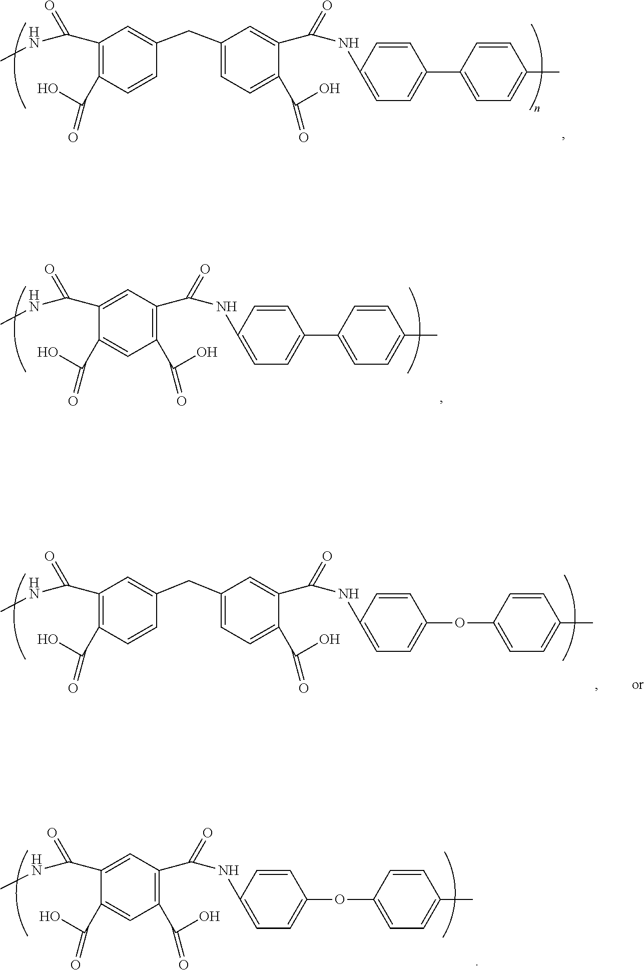

In one aspect, the precursor polyamic acid has the formula:

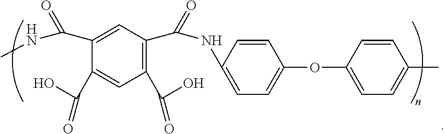

Any suitable polyimide film thickness is contemplated, which in general will result in a film or graphene material of less thickness due to combustion of organic and other material, as well as organization of the graphene layers. In one aspect, the layer of the polyimide or the polyimide substrate has an average thickness of 20-300 μm.

### B. Porous Graphene Electrode (First Electrode)

Graphene provides excellent performance characteristics for the ultrasound device, including mechanical robustness, flexibility, charge carrier concentration and mobility, thermal conductivity, and chemical resilience. Due to the graphitization of at least a portion of the polyimide, the porous graphene electrode has a variable porous structure, generally having an average pore diameter of less than 1 μm. The disclosed graphene may have a multi-scale structure, which enables good electrical properties. In some aspects, the porous graphene may have a structure including one or more of the following pore structures: macropores having an average pore size exceeding 50 nm; mesopores having an average pore size of 2-50 nm; micropores having an average pore size of 2 nm or less; and nanopores having an average pore size of less than 100 nm.

In some aspects, the nanopores of the graphene can provide for a high or even ultra high specific surface area, measured by the BET method which is known in the art. In one aspect, the nanopores of the porous graphene have a BET specific surface area of at least 100 m²/g. In one aspect, the nanopores of the porous graphene have a BET specific surface area of at least 150 m²/g. In one aspect, the nanopores of the porous graphene have a BET specific surface area of at least 200 m²/g. In one aspect, the nanopores of the porous graphene have a BET specific surface area of at least 225 m²/g. In one aspect, the nanopores of the porous graphene have a BET specific surface area of at least 250 m²/g. In one specific aspect, the nanopores of the porous graphene have a BET specific surface area of about 273 m²/g, “about” in this instance implying plus or minus 10 m²/g.

In further aspects, the nanopores of the porous graphene have a BET specific surface area of at least 300 m²/g. In a further aspect, the nanopores have a BET specific surface area of at least 400 m²/g. In a further aspect, the nanopores have a BET specific surface area of at least 500 m²/g. In a further aspect, the nanopores have a BET specific surface area of at least 600 m²/g. In a further aspect, the nanopores have a BET specific surface area of at least 700 m²/g. In a further aspect, the nanopores have a BET specific surface area of at least 800 m²/g. In a further aspect, the nanopores have a BET specific surface area of at least 900 m²/g. In a further aspect, the nanopores have a BET specific surface area of at least 1,000 m²/g. In a further aspect, the nanopores have a BET specific surface area of at least 1,200 m²/g. The upper limit for any of these threshold BET specific surface areas can vary, for example, 1,500 m²/g or in some aspects, 1,400 m²/g. In one aspect, the nanopores have a BET specific surface area of about 1,310 m²/g, “about” in this instance implying plus or minus 10 m²/g.

In a further aspect, the porous graphene exhibits a Horvath-Kawazoe pore volume of at least 0.2 cm³/g. For example, the graphene can exhibit a Horvath-Kawazoe pore volume of at least 0.25 cm³/g, at least 0.3 cm³/g, at least 0.35 cm³/g, at least 0.4 cm³/g, or at least 0.5 cm³/g. In one aspect, the porous graphene exhibits a Horvath-Kawazoe pore volume of at least 100 m²/g, e.g., about 105 m²/g, plus or minus 10 m²/g. The upper limit for any of these pore volumes can vary, e.g., 0.8 cm³/g, 0.7 cm³/g, 0.6 cm³/g, or 0.55 cm³/g. In some aspects, the described graphene exhibits a high degree of graphitization as is evident from a number of characteristics. In one aspect, the porous graphene has a mean graphene interlayer spacing of 0.35-0.45 nm. In one specific embodiment, the interlayer spacing of the graphene is 0.39 nm.

Thus, in one aspect, a disclosed embodiment of the porous graphene may have a structure including one or more of the following pore structures: macropores having an average pore size exceeding 50 nm; mesopores having an average pore size of 2-50 nm; micropores having an average pore size of 2 nm or less; and nanopores having an average pore size of less than 100 nm. In addition, this embodiment may have a BET specific surface area of at least 300 m²/g, or any of the BET surface areas described above.

The thickness of the porous graphene electrode in general depends on the thickness of the precursor polyimide film which is graphitized to form the porous graphene. In one aspect, the thickness of the graphene electrode is 20 μm-300 μm. In a further aspect, the thickness of the graphene electrode is 20 μm-250 μm. In a further aspect, the thickness of the graphene electrode is 20 μm-200 μm. In a further aspect, the thickness of the graphene electrode is 20 μm-150 μm. In a further aspect, the thickness of the graphene electrode is 20 μm-100 μm. In a further aspect, the thickness of the graphene electrode is 20 μm-75 μm. In a further aspect, the thickness of the graphene electrode is 30 μm-60 μm. In a further aspect, the thickness of the graphene electrode is 40 μm-60 μm. In a further aspect, the thickness of the graphene electrode is 45 μm-55 μm. Additional porous graphene materials are described in U.S. application Ser. Nos. 18/373,847, 18/532,214, and 18/406,060, each of which is incorporated into this application by reference in its entirety for its teachings or porous graphene and methods of making porous graphene.

Graphitization of the polyimide can be accomplished through a variety of contemplated methods. One example is laser irradiation of the polyimide. For example, irradiating can be performed with a CO₂ infrared laser, e.g., having a wavelength (λ) of 10.6 μm. In a specific aspect, irradiating can be performed at 1-12 Watts (e.g., 2-5 Watts), for example with 1,000 laser pulses per inch (PPI), and at a speed of 2-6 inches per second (e.g., 3-4 inches per second), for example with a CO₂ infrared laser, e.g., having a wavelength (λ) of 10.6 μm.

### C. Piezoelectric Material

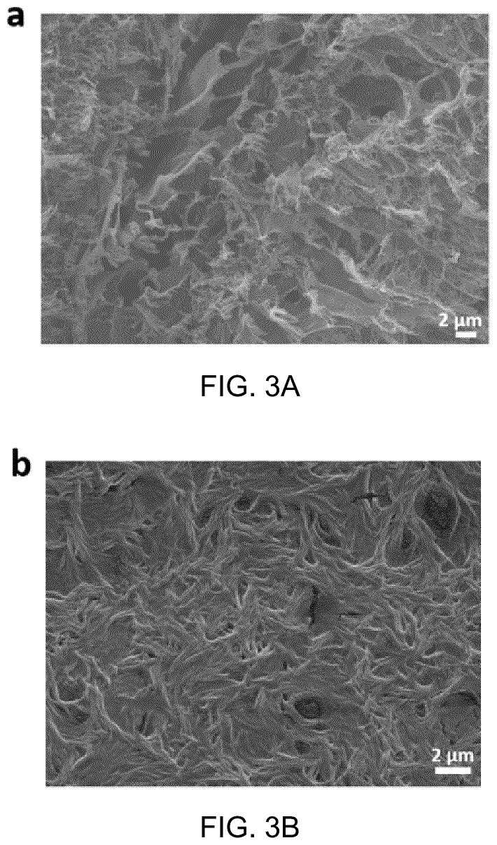

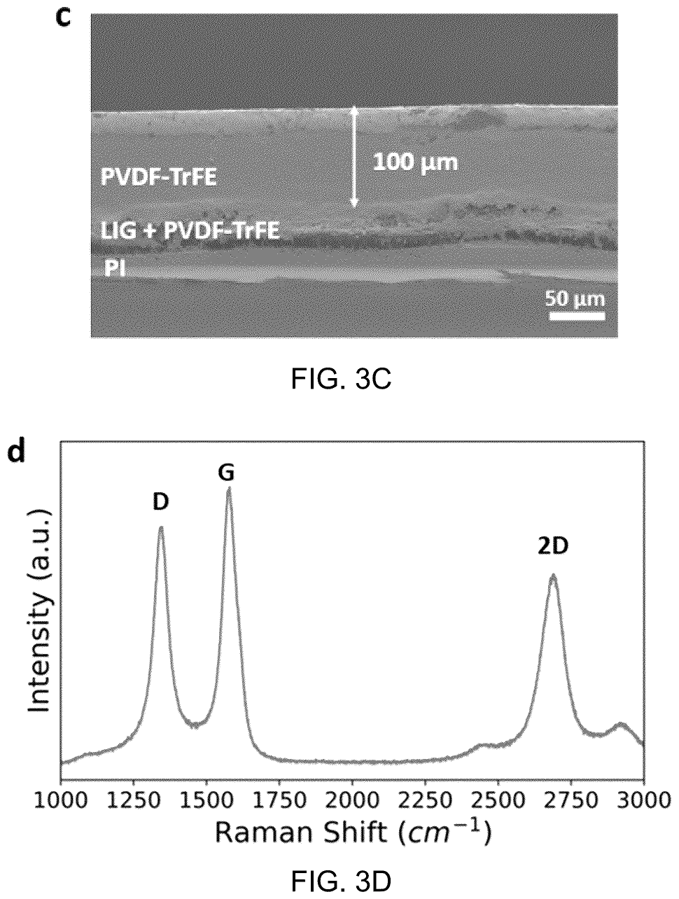

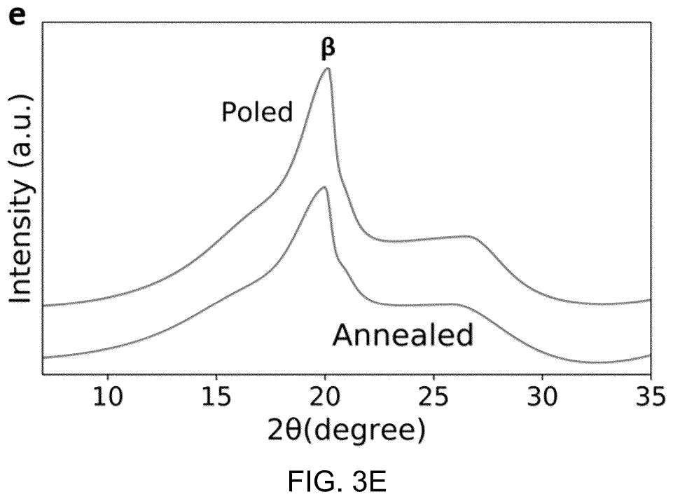

The ultrasound device further comprises a piezoelectric material disposed on the electrode and within at least a portion of the porous graphene. Owing to the highly porous nature of the graphene, the piezoelectric material, when applied to the electrode, can infiltrate the pores of the electrode, translating to a high specific surface area piezoelectric layer in the ultrasound device. Infiltration of the piezoelectric material into the pores of the graphene electrode can be determined by size exclusion chromatography (SEM), for example as shown in the cross-sectional image of FIG. 3C.

The layer of piezoelectric material may have a variety of thicknesses. Because the piezoelectric material embeds within the pores of the porous graphene electrode, in some aspects, there is no clear boundary line between the graphene electrode and the piezoelectric material. Thus, in some aspects, the composite electrode-piezoelectric material thickness can be characterized. In one aspect, after applying the piezoelectric material to the graphene electrode, the composite thickness can increase from 20-500% relative to the thickness of the starting graphene electrode layer. For instance, for a graphene electrode having a thickness of 20 μm-300 μm, the composite layer following piezoelectric material application can have an increased thickness of 24 μm-1,500 μm. In a further aspect, the composite thickness can increase from 100-200% relative to the thickness of the starting graphene electrode layer. For instance, for a graphene electrode having a thickness of 20 μm-300 μm, the composite layer following piezoelectric material application can have an increased thickness of 40 μm-600 μm.

A variety of piezoelectric materials are contemplated. In one aspect, the piezoelectric material is a polymer suitable for a deposition method such as spin coating or ink printing. In a further aspect, the piezoelectric material comprises a ferroelectric polymer. In one aspect, the piezoelectric material comprises a polymer or copolymer of vinyl fluoride. In a further aspect, the piezoelectric material comprises one or more of the following polymers or copolymers: polyvinylidene fluoride-trifluoroethylene (PVDF-TrFE), poly(vinylidene fluoride-trifluoroethylene-chlorofluoroethylene), P(VDF-TrFE-CFE), or polyvinylidene fluoride (PVDF). Non-limiting examples of PVDF copolymers include 60:40 (molar percent) PVDF-TrFE, 70:30 PVDF-TrFE, 80:20 PVDF-TrFE, and 90:10 PVDR-TrFE. In another aspect, piezoelectric pastes or slurries of various formulations of lead zirconate titanate (PZT) can be used.

The piezoelectric material can be applied to the porous graphene electrode through a variety of methods. In one aspect, the piezoelectric material can be dissolved or dispersed in a suitable solvent, for example, N—N dimethylformamide. The dispersion or solution of the piezoelectric material can then be applied through spin-coating with a mask overlay in some aspects, or in other aspects, direct printing of the piezoelectric material as an ink. Direct printing can be accomplished for example using commercially available printers (e.g., a Voltera Nova printer). In a further aspect, piezoelectric material can be applied to the graphene electrode through screen printing.

Fabrication of flexible ultrasound devices often requires the integration of a flexible piezoelectric material with conductive electrodes on a flexible substrate. Conventional piezoceramic materials often require high-temperature processes (over 700° C.) or methods such as mechanical dicing, which are not suitable for flexible polymeric substrates. Researchers have developed two main strategies to address this challenge. An advantage of the disclosed method is that a mechanically robust and flexible ultrasound device can be fabricated without using high temperatures or processes that are incompatible with flexible substrates.

After application of the piezoelectric material, the device can be annealed and in some aspects, poled. Poling is a step useful for aligning the dipoles within the piezoelectric material, which can enhance piezoelectric properties. In one aspect, poling can be accomplished by a corona poling process at a high direct current (DC) voltage, e.g., 8.5 kV, for a suitable time and generally at elevated temperatures (e.g., 50-100° C.). The electric field can be maintained while the temperature is gradually lowered to 40° C. over a suitable time. Suitable corona poling methods and devices for corona poling are known in the art. In another aspect, poling can be accomplished using contact poling process at high (DC) voltage, e.g., 30 kV, over a suitable time with or without compression, with or without elevated temperature, and either dry or submerged in silicone oil. Suitable contact poling methods and devices are known in the art.

### D. Second Electrode

In some aspects, the device comprises a second electrode formed on at least a portion of the piezoelectric material, i.e., with at least a portion of the piezoelectric material between the second electrode and the porous graphene or first electrode. In one aspect, the second electrode comprises silver, nickel, chromium, gold, aluminum, or a combination thereof. In a further aspect, the second electrode comprises gold. A second electrode material such as gold for instance can be applied to the piezoelectric material through a suitable technique such as sputter coating.

### E. Additional Components

In some aspects, the device comprises additional components useful for operating the device in a suitable ultrasound mode such as M-mode, two-dimensional (2D) mode, three-dimensional (3D) mode, or Doppler ultrasound. An electronics circuit may be connected to the first or second electrodes, or both, via a suitable electrical connector. The electronics circuit may be any suitable electronics circuit for collecting signals from the first and/or second electrodes. The electronics circuit may include an amplifier, such as a charge amplifier and/or a voltage amplifier.

A processor may be connected to the electronics circuit. The processor may receive signals detected by the transducer. The processor may be configured to filter the signals in some aspects. The processor may be in communication with a display, and may output an interface to the display. The interface may be generated based on the signals received from the transducer. The interface may display the signals received from the transducer or the filtered signals from the processor. The ultrasound device may be in electrical communication with a suitable power source such as a battery, e.g., for on-the-go applications such as assessment of soft tissue during running.

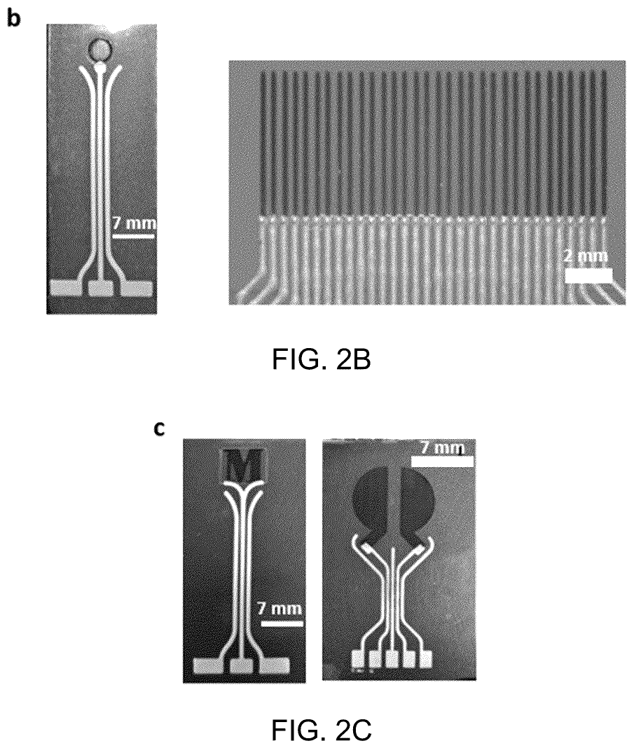

The ultrasound transducer may act as one or more of an acoustic sensor or an electric potential sensor. The transducer acting as an acoustic sensor may operate via a piezoresistive and/or optical force modality. In certain aspects, the transducer may have multiple functionalities. The function of the transducer may be determined based on how and/or whether a piezoelectric material of the transducer is polarized. Multiple transducers may be formed on the sheet, for example as an array, such as the 32-array transducer shown in FIG. 2B. Transducers sharing the same substrate may have the same or different sensor functions.

The transducer may act as a sensor for photoacoustic signals, where the ultrasound waves are generated by absorption of light of one or more wavelengths and subsequent thermoelastic expansion of features within the medium that contain light-absorbing chromophores to facilitate photoacoustic imaging. In certain aspects, the porous graphene layer within the transducer can be optically excited to act as the source of ultrasound pulses by virtue of the photoacoustic phenomenon and corresponding echo signals can be detected by the same transducer to facilitate ultrasound imaging.

The transducer may be covered or surrounded by an insulating layer (also referred to as “passivation layer”). One example of such a material is parylene. In certain aspects, the insulating layer is formed of the same material as the substrate. In other aspects, the insulating layer may be made of another material. For example, in some aspects, the insulating layer is made of a material that protects the second electrode or another uppermost electrode. In other aspects, the insulating layer is made of a material that has a shielding effect from electric or acoustic signals. In yet other embodiments, the insulating layer may be optically transparent or translucent.

The transducer may be surrounded by a shield, such as a metal shield around an exterior of the transducer. Alternatively, the shield may extend over the second electrode, another uppermost electrode (when present) or the insulting layer (when present). The metal shield may or may not be placed against the skin of the user. Multiple transducers in an array configuration may also share a ground connection.

### II. Ultrasound Device Applications

In one aspect, the disclosed ultrasound device is useful for assessing an anatomical structure in a subject. In one aspect, the method comprises obtaining at least one ultrasound image from an ultrasound device applied to a target skin area of the subject, wherein the ultrasound device comprises: (a) a first electrode comprising porous graphene, the first electrode being disposed within or on at least a portion of a substrate comprising a polyimide which has at least one aromatic ring; and (b) a piezoelectric material disposed on the first electrode and within at least a portion of the porous graphene. The ultrasound device used in the assessment method may have any of the compositions or characteristics described above.

In one aspect, a system for assessing and monitoring an anatomical structure of a subject can comprise at least one ultrasound device (e.g., a patch) attached to a subject, wherein the device comprises one or more ultrasound sensors, a communication system, and an electric board for ultrasound transmission and/or reception. The ultrasound device can generate at least one ultrasound image in one or more modes such as M-mode, two-dimensional (2D) mode, three-dimensional (3D) mode, or Doppler ultrasound mode. In a further aspect, the system can comprise a server having a cloud system for processing the at least one ultrasound image. In some aspects, processing can include one or more analytical tools such as radon transformation, higher-order spectra (HOS) techniques, or active contour model.

While ultrasound echoes can be acquired continuously and images generated continually and stored throughout the time that the ultrasound sensor is being applied, the image output can also be stored in the form of a still image or moving images in video format (“cine”) at the discretion of the operator. The duration of the “moving” image is typically up to a few or several seconds that is deemed sufficient to depict the phasic motion of the structure of interest. A single still ultrasound image can be a stored 2D image of the structure captured at one finite period in time. Alternatively, a still image can also capture one-dimensional spatial or Doppler-derived velocity information that is acquired over a time period, typically a few or several seconds, that is deemed sufficient to depict the phasic motion of the structure of interest. In particular, M-mode ultrasound depicts one-dimensional spatial information on the y-axis against time on the x-axis, while spectral Doppler ultrasound depicts velocity information on the y-axis against time on the x-axis.

In one aspect, the ultrasound device is flexible and conforms to the surface of the subject's skin. In one another aspect, the ultrasound device can be modified and adapted to be attached to and conform with the surfaces of internal body cavities of a subject. In a further aspect, the ultrasound device can be modified and adapted to operate as an implantable sensor.

The disclosed device may be used for any suitable ultrasound application, including applications benefitting from a wearable ultrasound device. One example is the dynamic assessment of musculoskeletal tissues using a compact, portable, and wearable ultrasound device, for example using the device in M-mode and/or 2D brightness-mode (B-mode). Another example is robust myography during physical activity and movement. For example, the wearable application of the device enables real-time examination of muscle quality, tissue composition fascial gliding, cross-sectional area, fascicle pennation angle, and contraction velocity, for example during aerobic exercise or weight training. Such an application in some aspects can guide training or recovery from musculoskeletal injury.

Kinematics and kinetics can also be observed as a means for characterizing muscle tissue properties. For instance, the ultrasound device can be used to correlate joint angle and joint torque during functional electric stimulation. In another example, quantification of the echogenicity of the Vastus Lateralis can be used to estimate the muscle contribution to joint force during a weight training movement such as isometric squats or cyclic knee extensions. M-mode imaging may also be used for dynamic assessment of muscle-activation synergy. Several single-element ultrasound transducers can be placed at different anatomical sites for M-mode imaging, significantly simplifying the instrumentation needs. In some aspects, multiple single-element transducers are equivalent in performance relative to a conventional clinical ultrasound system for depths up to 4 cm.

Performance of the described devices was found to be excellent. In some aspects, the devices are mechanically robust, since it was observed that the porous graphene can be subjected to 10,000 cycles of bending and twisting strains which maintaining high electrical conductivity and mechanical flexibility. The ultrasound devices produced a pulse-echo signal with a signal to noise ratio (SNR) of greater than ten. The devices can also achieve dynamic M-mode imaging of acoustically reflective interfaces, similar to conventional but largely inflexible piezo-ceramic devices.

In some aspects, a hands-free ultrasound transducer in a patch type configuration can sustain prolonged probe-skin contact without impairing physical movement and while maintaining its electromechanical characteristics through extensive bending and twisting. When integrated with miniaturized electronics, the ultrasound device can allow clinicians to visualize mobility of deep scar tissues, soft tissue-to-bone interfaces, tissue adherences, gliding between muscle compartments, and volume changes. Post-injury assessment may also include evaluation of large hematoma, contusions, or behavior of tissues during walking tasks following penetrating trauma from wounds. Addition of multiple views within the same muscle group, or antagonistic muscle groups in the same limb, can add clinical information to benefit the clinical assessment of and rehabilitation treatment of injured subjects. Subjects who suffer tissue volume loss after surgical debridement and who subsequently require prosthetics may benefit from a deeper study of tissue behavior during rehabilitative exercises with and without prosthetic devices. The technology can be applied to a variety of injuries including those occurring from sports or falls.

### Examples

The following examples further illustrate this disclosure. The scope of the disclosure and claims is not limited by the scope of the following examples.

### I. Ultrasound Transducer

### A. Fabrication Method

To synthesize the composite laser-induced graphene/polyvinylidene fluoride-trifluoroethylene (LIG/PVDF-TrFE), a combination of photothermal laser manufacturing and printing techniques were employed (FIG. 2A). LIG was synthesized as a functional working electrode from a polyimide film using photothermal laser treatment. The polyimide had the following structure:

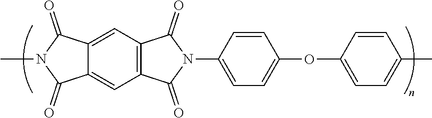

In contrast to conventional methods such as chemical vapor deposition, the use of photothermal laser processing offers a quicker and simpler approach, enabling rapid patterning and targeted graphitization. The intensity of the light used in this process was specifically adjusted for the material undergoing laser treatment, controlling the local temperature through various operational parameters, including laser power, laser speed, and pulses per inch (PPI). This technique involved the use of a 10.6 μm CO₂ laser, where the area of focus reaches temperatures exceeding 2500° C. A 10.6 μm CO₂ laser-cutter system (Universal, VLS2.30) was used for fabricating the LIG at a laser power of 4.2 W. The pulse duration and scan speed were fixed at 14 μs, and 3.5 in s⁻¹, respectively, and the laser treatment was performed under ambient conditions. The heat in the process breaks down the covalent bonds in the polyimide, leading to the release of H₂, N₂, and O₂ gases. As a result of the high-temperature process, the evaporated gases create a variety of macro- and mesopores within the graphene structure.

Conductive silver ink was used to print interconnects and contact pads using a Voltera printer. This was followed by the application of UV-cure silicone as a shadow mask and deposition of piezoelectric PVDF-TrFE (˜100 μm thick) as the piezoelectric material. The printed PVDF-TrFE layer underwent drying and annealing at 140° C. for 20 minutes. The printing and drying steps of PVDF-TrFE ensure the formation of the LIG/PVDF-TrFE composite, characterized by high uniformity and an increased surface area of the PVDF-TrFE. The increased surface area of PVDF-TrFE is likely attributed to the high specific surface area and three-dimensional porous structures of LIG, enabling infiltration of PVDF-TrFE into the interconnected pores of LIG. The enhanced surface area of the piezoelectric PVDF-TrFE improves the piezoelectricity and the functional performance of the material in ultrasound applications.

After the annealing process, the PVDF-TrFE was subjected to a corona poling process at a high direct current (DC) voltage of 8.5 kV for 60 minutes at a temperature of 80° C. The electric field was maintained while the temperature was gradually lowered to 40° C. over 100 minutes. This step aligns the dipoles within the material, enhancing its piezoelectric properties. Finally, gold (Au) was coated (˜60 nm thick) through a sputter coater. This coating process was performed twice, each time at a current of 3 mA for 300 seconds. To complete the process, a 1 μm thick parylene film was deposited on the device for passivation, using a parylene coater.

FIG. 2B shows a photograph of a single-element LIG/PVDF-TrFE transducer and a microscope image of a 32-array transducer. The scalability and precise patterning capability of the described photothermal laser manufacturing technology allow for the creation of array transducers with a pitch of ˜350 μm, for example. Ultrasound transducers of various sizes and patterns can be prepared on demand by computer-controlled patterning in the air at room temperature during the photothermal laser process, as evidenced by the photographs of transducers having different patterns in FIG. 2C.

### B. Characterization

The morphological and crystalline structure of the LIG/PVDF-TrFE composite was analyzed using SEM, Raman, and XRD. The SEM image from FIG. 3A reveals the porous structure of LIG with an average diameter in the sub-micrometer range. The SEM of PVDF-TrFE coated on LIG is displayed in FIG. 3B. The fibrous pattern is due to the fabrication method using Voltera printing with a nozzle. The cross-sectional SEM image reveals a ˜50 μm thick LIG with a uniformly coated 100 μm thick PVDF-TrFE layer on top (FIG. 3C). The average thickness of the composite is dependent on the number of PVDF-TrFE layers applied, increasing from 50 μm for bare LIG to 150 μm for LIG/PVDF-TrFE after 15 passes. Cross-sectional SEM images indicate that for up to 6 passes, the PVDF-TrFE infiltrates the porous structure of LIG, forming a nanocomposite with PVDF-TrFE embedded within LIG. Beyond 6 passes, PVDF-TrFE begins to form a uniform layer atop the LIG.

Raman spectroscopy analysis was performed to confirm graphene formation from the polyimide precursor and analyze its crystalline structure. FIG. 3D shows three prominent peaks: D (≈1346 cm⁻¹), G (≈1576 cm⁻¹), and 2D (≈2688 cm⁻¹), indicating the formation of graphene through photothermal laser treatment. The crystalline properties of PVDF-TrFE after annealing and poling were characterized by x-ray diffraction (XRD). FIG. 3E shows a sharp peak at 20.12° which represents the Bragg diffraction of (110)/(200) of the β-phase, indicative of the ferroelectric β-phase with an all-trans conformation. After poling, the peak at 20.12° became sharper, indicating an increase in piezoelectric properties.

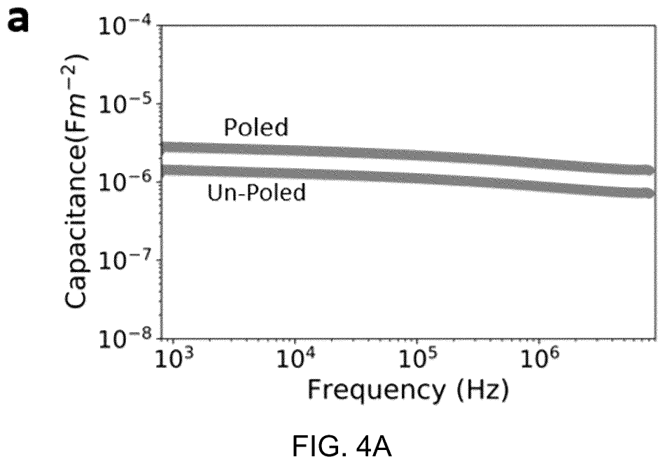

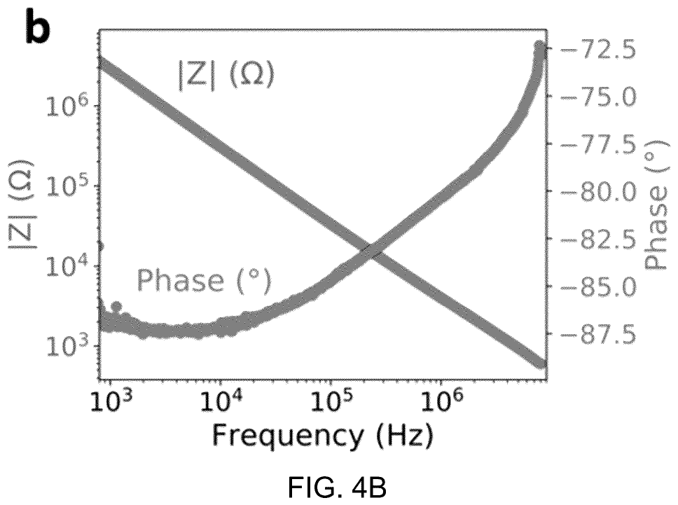

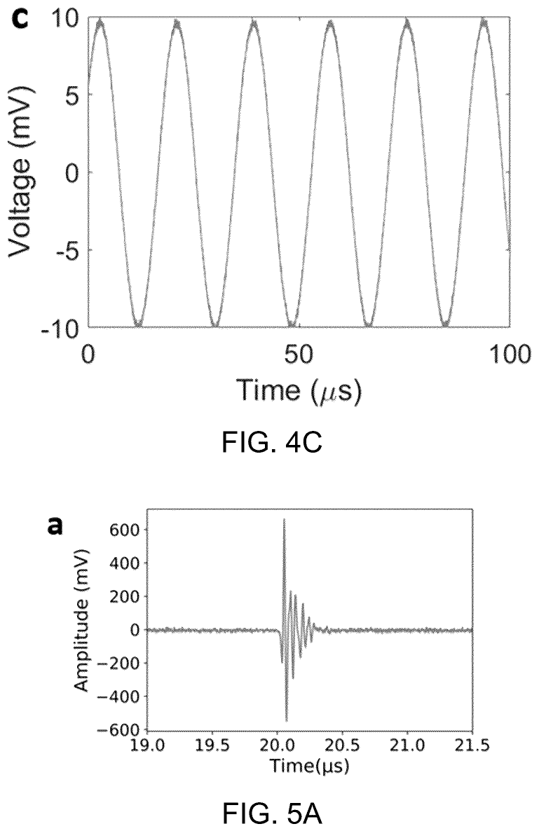

To confirm the successful poling, the electrical and piezoelectric properties of the LIG/PVDF-TrFE were investigated by analyzing capacitance, and by conducting a compression piezo response test. FIG. 4A compares the capacitance of the transducer at different frequencies before and after poling, showing increased capacitance in the poled device. The increase indicates that poling was successful. Capacitance is directly related to the dielectric constant of the material, (C=εᵣε₀A/d), where εᵣ is the relative permittivity or dielectric constant of the material, ε is the permittivity of free space (a constant approximately equal to 8.854×10⁻¹² farad per meter), A is the area of the plates of the capacitor, and d is the distance between the two plates). The frequency dependence of electric impedance and phase of a transducer is shown in FIG. 4B. The successful poling process was confirmed by conducting a compression piezo response test and measuring the output voltage of LIG/PVDF-TrFE under compression test with respect to time. FIG. 4C shows the large voltage amplitude of 20 mV after poling the transducer, which gives the high piezoelectric coefficient (d33) of 99.41 pm/V.

### C. Ultrasound Transducer Performance

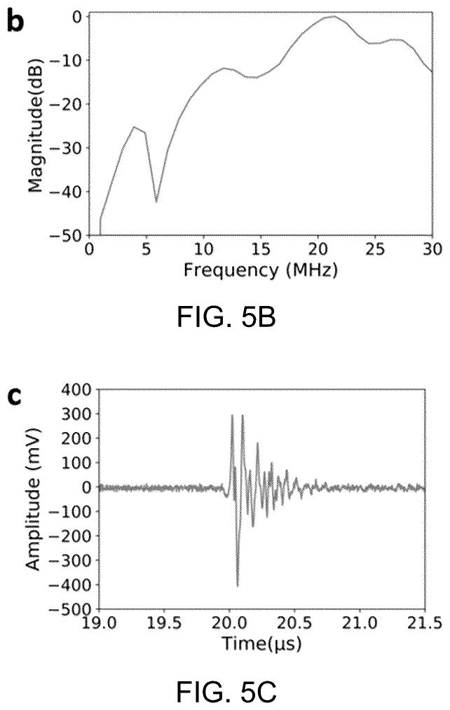

A pulse-echo test was performed in water to evaluate the performance of the LIG/PVDF-TrFE transducer. FIG. 5A displays the pulse-echo signal captured by the transducer. The voltage signal amplitude of the transducer exhibits a substantial peak-to-peak output voltage of 1.2 V, coupled with a high signal-to-noise ratio (SNR) of 109.45. To assess the impact of integrating LIG with PVDF-TrFE, output data were collected from a ultrasound transducer based on Ag/PVDF-TrFE. For this comparative transducer, the PVDF-TrFE was only positioned atop the electrode, which results in a lower effective surface area compared to that in the LIG/PVDF-TrFE integrated composite. The peak-to-peak output voltage of the Ag/PVDF-TrFE UST was measured to be 0.7 V with a SNR of 44 (FIG. 5C).

The significantly larger SNR in the LIG/PVDF-TrFE UST, exceeding more than twice that of the Ag/PVDF-TrFE, can likely be attributed to the enhanced piezoelectricity of the PVDF-TrFE. This enhancement is likely a result of its higher effective surface area when embedded in the interconnected pores of the graphene. The output voltage time-domain waveform was analyzed alongside the frequency-domain spectrum. A Fast Fourier Transform (FFT) was used to convert the time-based voltage signal into the frequency domain. This conversion yielded a plot of amplitude in decibels against frequency in MHz, as depicted in FIG. 5B. The transducer, integrating LIG and PVDF-TrFE, consistently exhibited a strong and distinct signal with a central frequency of approximately 21.48 MHz and a bandwidth of 5.28 MHz. The high central frequency of the transducer highlights its ability to effectively detect a broad range of ultrasound frequencies. Such a high central frequency in the ultrasound transducer demonstrates potential for significantly improved resolution in ultrasound imaging relative to commercially available ultrasound transducers.

Features and advantages of this disclosure are apparent from the detailed specification, and the claims cover all such features and advantages. Numerous variations will occur to those skilled in the art, and any variations equivalent to those described in this disclosure fall within the scope of this disclosure. Those skilled in the art will appreciate that the conception upon which this disclosure is based may be used as a basis for designing other compositions and methods for carrying out the several purposes of this disclosure. As a result, the claims should not be considered as limited by the description or examples.

What is claimed is:

1. An ultrasound device comprising:
a) a first electrode comprising porous graphene, the first electrode being disposed within or on at least a portion of a substrate comprising a polyimide which has at least one aromatic ring; and
b) a piezoelectric material disposed on the first electrode and within at least a portion of the porous graphene.

2. The device of claim 1, wherein the substrate is flexible.

3. The device of claim 1, wherein the porous graphene has an average pore diameter of 1 μm or less.

4. The device of claim 1, wherein the polyimide has the structure of formula I or II: 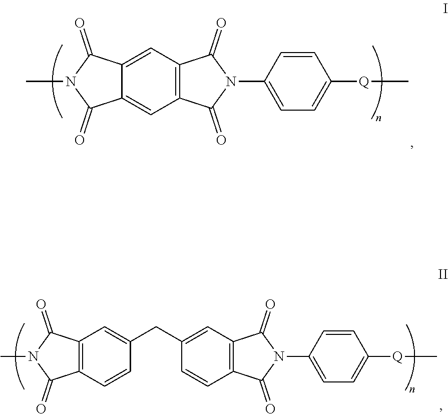
wherein n is an integer of at least two, and Q has the structure A or B:

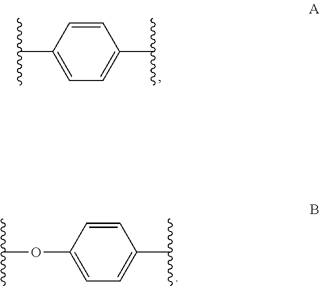 [SMILES: *c1ccc(*)cc1 ; *Oc1ccc(*)cc1]

5. The device of claim 1, wherein the piezoelectric material comprises a polymer of vinyl fluoride.

6. The device of claim 5, wherein the polymer of vinyl fluoride is polyvinylidene fluoride (PVDF), polyvinylidene fluoride-trifluoroethylene (PVDF-TrFE), poly(vinylidene fluoride-trifluoroethylene-chlorofluoroethylene) (PVDF-TrFE-CFE), or a combination thereof.

7. The device of claim 1, further comprising a second electrode disposed on at least a portion of the piezoelectric material.

8. The device of claim 7, wherein the second electrode comprises silver, nickel, chromium, gold, aluminum, or a combination thereof.

9. A method of making a device comprising:
a) graphitizing at least a portion of a substrate comprising a polyimide having at least one aromatic ring to form a first electrode comprising porous graphene disposed within or on the substrate; and
b) applying a piezoelectric material to at least a portion of the first electrode.

10. The method of claim 9, wherein the substrate is flexible.

11. The method of claim 9, wherein the porous graphene has an average pore diameter of 1 μm or less.

12. The method of claim 9, wherein the polyimide has the structure of formula I or II: 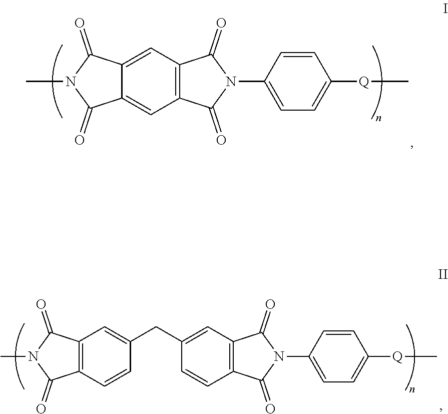
wherein n is an integer of at least two, and Q has the structure A or B:

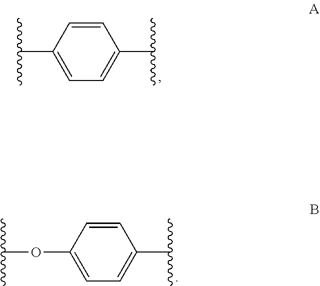 [SMILES: *c1ccc(*)cc1 ; *Oc1ccc(*)cc1]

13. The method of claim 9, further comprising preparing the polyimide by thermally imidizing a precursor polyamic acid.

14. The method of claim 9, wherein the polyimide is graphitized by irradiating the polyimide with an infrared laser under conditions sufficient to form the porous graphene.

15. The method of claim 9, wherein the piezoelectric material comprises a polymer of vinyl fluoride.

16. The method of claim 15, wherein the polymer of vinyl fluoride is polyvinylidene fluoride (PVDF), polyvinylidene fluoride-trifluoroethylene (PVDF-TrFE), poly(vinylidene fluoride-trifluoroethylene-chlorofluoroethylene) (PVDF-TrFE-CFE), or a combination thereof.

17. The method of claim 9, further comprising forming a second electrode on at least a portion of the piezoelectric material.

18. The method of claim 17, wherein the second electrode comprises silver, nickel, chromium, gold, aluminum, or a combination thereof.

19. An ultrasound device prepared by the method of claim 9.

20. A method of assessing an anatomical structure in a subject, comprising obtaining at least one ultrasound image from an ultrasound device applied to a target area of the subject, wherein the ultrasound device comprises:
a) a first electrode comprising porous graphene, the first electrode being disposed within or on at least a portion of a substrate comprising a polyimide which has at least one aromatic ring; and
b) a piezoelectric material disposed on the first electrode and within at least a portion of the porous graphene.
